(19) United States
(12) Patent Application Publication
(10) Pub. No.: US 2005/0175575 A1
(43) Pub. Date: Aug. 11, 2005

(54) Shaving compositions

(76) Inventors: Yun Xu, Andover, MA (US); Stephen Thong, Needham, MA (US); Gurusamy Manivannan, Maryland Heights, MO (US); David T. Callaghan, Foxborough, MA (US)

Correspondence Address: FISH & RICHARDSON PC, 225 FRANKLIN ST, BOSTON, MA, 02110, US

(21) Appl. No.: 10/777,009

(22) Filed: Feb. 11, 2004

(51) Int. Cl. A61K 7/15

(52) U.S. Cl. 424/73

(57) ABSTRACT

The present invention is directed to shaving compositions that include water insoluble lubricating polymer particles and/or one or more water soluble polymers. Some preferred compositions include a lubricating agent that contains water insoluble lubricating polymer particles and a blend of two or more water soluble polymers having different molecular weights. The invention is also directed to methods of shaving in which a shaving composition of the present invention is applied to an area of skin, then said area is shaved, preferably with a wet razor.

### TECHNICAL FIELD

This invention relates to shaving compositions and methods, and more particularly to shaving compositions for use in wet shaving.

### BACKGROUND

Currently, a widely used form of shaving preparation is the type referred to as a post-foaming shave gel. These post-foaming shave gels are now well-known and have been described, for example, in U.S. Pat. No. 2,995,521 (Bluard), U.S. Pat. No. 3,541,581 (Monson), U.S. Pat. No. 4,405,489 (Sisbarro), U.S. Pat. No. 4,528,111 (Su), U.S. Pat. No. 4,651,503 (Anderson), U.S. Pat. No. 5,248,495 (Patterson), U.S. Pat. No. 5,308,643 (Osipow), U.S. Pat. No. 5,326,556 (Barnet), U.S. Pat. No. 5,500,211 (George), U.S. Pat. No. 5,560,859 (Hartmann) and U.S. Pat. No. 5,858,343 (Szymczak). Such compositions generally take the form of an oil-in-water emulsion in which the post-foaming agent, generally a volatile (i.e., low boiling point) aliphatic hydrocarbon, is solubilized in the oil phase, and the water phase comprises a water-dispersible soap or interrupted soap component. The product is generally packaged in an aerosol container with a barrier, such as a piston or collapsible bag, to separate the post-foaming gel from the propellant required for expulsion of the product. The product is dispensed as a clear, translucent or opaque gel that is substantially free from foaming until it is spread over the skin, at which time it produces a foam lather generated by the volatilization of the volatile hydrocarbon foaming agent. Other shaving preparations are provided as foams or as non-foaming, non-aerosol gels.

These shaving preparations are formulated to enhance shaving performance and improve comfort during shaving, in part by providing lubricity and glide and thereby reducing the friction between the razor cartridge and the user's skin. Some shaving preparations include water-insoluble particles to enhance the performance of the preparations. For example, U.S. Pat. No. 5,587,156 discloses shaving preparations that contain water-insoluble particles, e.g., polytetrafluoroethylene (PTFE) particles, in an effective amount to produce physical micro-support for the blade during shaving, which is said to prevent nicks and cuts. In EP 0829259, powders such as micronized PTFE are added to shaving creams and gels to provide exfoliation. It is also known to include perfluorocarbon resins in moisturizing compositions as a dry lubricant, e.g., as described in U.S. Pat. No. 3,932,614.

### SUMMARY

The inventors have found that the lubricity and glide provided by a shaving preparation can be significantly improved by including water-insoluble lubricating polymer particles that include polytetrafluoroethlyene (PTFE) in the shaving preparation. Lubricity and glide are generally improved by including in the shaving preparation one or more water soluble polymers, in some cases a blend of two or more water soluble polymers having different molecular weights.

In one aspect, the invention features a method of shaving comprising applying to an area of skin a shaving composition and shaving said area of skin, wherein the shaving composition comprises an aqueous solution including a cleansing or conditioning agent for hair or skin, water-insoluble lubricating polymer particles comprising polytetrafluoroethylene, and a water soluble polymer, i.e., a polymer that will reduce the coefficient of friction between a razor cartridge and the user's skin, as compared to the coefficient of friction that would be obtained when using the same shaving composition without the water soluble polymer.

In another aspect, the invention features a shaving composition including water, a cleansing or conditioning agent for hair or skin, and a lubricating agent. The lubricating agent includes (a) lubricating polymer particles comprising PTFE, and (b) a water soluble polymer.

Some implementations of the method and shaving preparations described above may include one or more of the following features. The water soluble polymer may include polyethylene oxide. The shaving composition may be in the form of a shaving lotion, cream, foam or gel. The lubricating polymer particles and water soluble polymer may be provided in a ratio of about 0.25:1 to about 3:1. The shaving composition may include from about 0.005% to 2% of the lubricating polymer particles, and from about 0.01 to 2.0% of the water soluble polymer. The polymer particles may have an average particle size of from about 1 μm to 100 μm. The shaving composition may further include from about 3 to 25% of a water dispersible or water soluble surface active agent. The cleansing or conditioning agent may be selected from the group consisting of beard wetting agents, skin conditioning agents, cleansing agents, lathering agents, emollients, humectants, skin freshening and soothing agents, silicones, vitamins, and water dispersible or soluble surface active agents. The water soluble polymer may be selected from the group consisting of polyethers, vinyl polymers, vinyl copolymers and terpolymers, natural polymers, and mixtures thereof. The water soluble polymer may include a blend of polyethylene oxide and a water soluble natural or synthetic gum. The water soluble polymer may have a molecular weight of at least one million. The shaving composition may also include a volatile self-foaming agent.

In a further aspect, the invention includes a shaving composition including water, a cleansing or conditioning agent for hair or skin, and a lubricating agent including (a) from about 0.005% to 2% of lubricating polymer particles comprising PTFE, and (b) from about 0.01 to 2.0% of a water soluble polymer selected from the group consisting of polyethers, vinyl polymers, vinyl copolymers and terpolymers, natural polymers, and mixtures thereof.

The invention also features a shaving composition including water, a cleansing or conditioning agent for hair or skins selected from the group consisting of beard wetting agents, skin conditioning agents, cleansing agents, lathering agents, emollients, humectants, skin freshening and soothing agents, silicones, vitamins, and water dispersible or soluble surface active agents, and a lubricating agent including (a) from about 0.005% to 2% of lubricating polymer particles comprising PTFE, and (b) from about 0.01 to 2.0% a water soluble polymer, wherein components (a) and (b) are provided in a ratio of about 0.25:1 to about 3:1.

As will be discussed below, in some preferred compositions, the lubricating agent, and particularly the water soluble polymer, is substantially free of anionic polymers, i.e., the lubricating agent contains less than 0.5%, preferably less than 0.2%, more preferably less than 0.1% of anionic polymer (by weight of the lubricating agent). The phrase “anionic polymers,” as used herein, includes polymers that are anionic as supplied and polymers that can be converted into anionic polymers under alkaline conditions, such as carboxypolymethylene. Preferably, the lubricating agent, particularly the water-soluble polymer, and the shaving composition itself, is free of anionic polymer. Preferred lubricating agents, and particularly preferred water soluble polymers, for use in such compositions are generally non-ionic.

In other aspects, the invention features other combinations of the preferred features discussed herein.

The details of one or more embodiments of the invention are set forth in the accompanying drawings and the description below. Other features and advantages of the invention will be apparent from the description and drawings, and from the claims.

### DETAILED DESCRIPTION

The shaving compositions of the present invention are aqueous compositions and may be in the form of liquids, lotions, foams, or post-foaming or non-foaming gels. One aspect of the present invention embraces a shaving composition comprising water, a cleansing or conditioning agent for hair or skin, and a lubricating agent including lubricious water insoluble polymer particles and at least one water soluble polymer. Preferred shaving compositions include a lubricating agent containing polymer particles that include PTFE, and one or more water soluble polymer(s). Preferred shaving compositions exhibit excellent lubricity and glide, and provide a close, comfortable shave and desirable post shave skin attributes such as smooth skin feel. Some preferred compositions may also provide mild skin exfoliation. The water-insoluble polymer particles may also provide aesthetic appeal in some compositions.

Suitable PTFE-containing polymer particles, when incorporated in a shaving preparation, will reduce the measured coefficient of friction between the razor cartridge and the user's skin, as compared to the coefficient of friction that would be obtained when using the same shaving preparation without the polymer particles. Alone or preferably in combination with water soluble polymer(s), it can also provide skin conditioning function leading to smooth, tack-free, silky feel skin after shave. For optimal glide, lubricity and skin conditioning, it is generally preferred that the particles have an average particle size of from about 1 μm to 100 μm, more preferably 5 μm to 15 μm. However, if exfoliation is desired, it may be desirable to include particles having a larger particle size, e.g., 100 μm to 1000 μm, preferably 200 μm to 500 μm. The particles may be colored if desired to provide an aesthetic effect. This may be particularly desirable in a clear or semi-transparent gel product.

Suitable polytetrafluoroethylene particles include those commercially available from Micro Powders, Inc. under the tradename MICROSLIP. The particles may be micronized. Preferably, the particles are comprised of 100% PTFE. However, if desired, the particles may contain other polymers or additives.

Preferably the polymer particles are dispersed uniformly throughout the shaving preparation. The particles may be used in any amount that improves lubricity and glide without undesirably compromising the other attributes of the shaving preparation. Generally, the composition will include from about 0.005 to about 2% of the particles. In some cases, it may be desirable to use less than 0.1% of the particles, e.g., 0.05% or less.

Suitable water soluble polymers, when incorporated in a shaving preparation, will reduce the coefficient of friction between the razor cartridge and the user's skin, as compared to the coefficient of friction that would be obtained when using the same shaving preparation without the water soluble polymer. Some water soluble polymers, when incorporated in a shaving preparation, will enhance the aesthetics and shave performance of the lather, such as the lather creaminess and cushioning. In many cases, suitable water soluble polymers also serve as thickening agents and help to form the gel structure, when the shaving preparation is provided in the form of a shave gel.

Suitable water soluble polymers or combinations of water soluble polymers that may be advantageously utilized in the shaving compositions of the present invention may be readily found by incorporating candidate polymers or polymer combinations into a preselected shaving composition, then measuring the coefficient of friction of that shaving composition. Any water soluble polymer or polymer combination may be utilized which, when used in combination with the PTFE particles, reduces the coefficient of friction of the shaving composition relative to the coefficient of friction of the shaving composition without the water soluble polymer(s). Preferably, the water soluble polymer, when used in combination with the PTFE particles, significantly reduces the coefficient of friction of the shaving composition relative to the coefficient of friction of the shaving composition without the water soluble polymer(s). It is also preferred that the water soluble polymer not interfere with or deleteriously affect other desirable aesthetic or performance attributes of the shaving composition. For example, it is generally preferred that the water soluble polymer is substantially free of anionic polymers, as such polymers may deleteriously interact with the base used to form a soap in some shaving compositions, which in turn may have a negative effect on gel stability and/or aesthetic characteristics of the compositions. It is generally preferred that the water soluble polymer is non-ionic.

Candidate water soluble polymers may be selected from, but are not limited to, those falling within the following polymer types:

High molecular weight water soluble polyethers, such as those of the formula R—(OCH₂—CH₂)ₙ—R′, where R and R′ may be any moiety which does not cause the polymer to become insoluble in water and n is typically 20,000 or more. For example, R and R′ independently may be selected from H, OH, CH₃, CONH2, and COOH. Polyethylene oxide of molecular weight one million or more is preferred.

High molecular weight water soluble vinyl polymers, such as those of the formula —(CH₂—CRR′)ₙ—, where R and R′ may be any moiety which can confer water solubility and n is an integer sufficient to provide a molecular weight of one million or higher. For example, R may be H or CH₃ and R′ may be OH, CONH2, pyrrolidone, or COOH. These polymers include polyacrylamides, polyvinylpyrrolidones, polyacrylic acids, polymethacrylic acids and copolymers and terpolymers thereof.

High molecular weight water soluble vinyl copolymers and terpolymers, such as those of the formula —(CH₂—CR₁R₂)ₙ—CH₂—CR₃R₄)ₘ—(CH₂—CR₅R₆)ₚ—, where R₁ to R₆ independently may be selected from any moiety such that the combination of R₁ to R₆ confers water solubility, and where n, m and p are zero or an integer such that n+m+p is sufficient to provide a molecular weight of one million or higher. In other words, at least one of R₁ to R₆ must be water soluble and the total number of such water soluble groups in the molecule must be such as to render the polymer water soluble. Examples include poly(acrylamide-co-acrylic acid), poly(acrylamide-co-2-acrylamido-2-methyl-1-propanesulfonic acid), poly(acrylamide-co-3-acrylamido-3-methylbutanoic acid), poly(methacrylic acid-co-acrylamide), and poly(N-vinylpyrrolidone-co-2-dimethylaminoethyl methacrylate).

High molecular weight natural polymers such as cellulose ethers, for example hydroxyethyl cellulose, carboxymethyl or methyl cellulose; protein-based polymers such as wheat proteins and collagen; starch and fermentation products such as Xanthan Gum, starch blends, and lactic acid polymers; exudate and vegetable gums such as Guar Gum and Gum Arabic; and marine polymers such as alginates and carrageenan.

One particularly advantageous combination of polymers which may be utilized in shaving compositions includes polyethylene oxide and a water soluble natural or synthetic gum. It has been found that polyethylene oxide and natural or synthetic gum, as described above, interact synergistically to substantially increase the stress ratio of a shaving composition, thereby reducing the coefficient of friction between the razor cartridge and the skin.

In some cases, the lubricating agent may include a blend of two or more water soluble polymers having different molecular weight distributions, for example a blend of two or more polyethylene oxides having different molecular weight distributions.

Suitable polyethylene oxide blends include one or more polyethylene oxides with a relatively high molecular weight of about two million or higher, typically up to about 5 million. Suitable relatively high molecular weight polyethylene oxides include, for example, one or more of the following: PEG-45M (WSR N-60K (Union Carbide; M. Wt. @2,000,000)), PEG-90M (WSR-301 (Union Carbide; M. Wt. @4,000,000)), PEG-115M (Polyox Coagulant (Union Carbide; M. Wt. @5,000,000). The relatively high molecular weight polyethylene oxide is blended with one or more relatively lower molecular weight polyethylene oxides, e.g., of molecular weight less than or equal to one million. Suitable relatively lower molecular weight polyethylene oxides include, for example, one or more of the following: PEG-23M (WSR N-12K (Union Carbide; M. Wt. @1,000,000) and PEG-7M (WSR N-750 (Union Carbide, M. Wt.@300,000)). The polyethylene oxides may be used in any desired ratio.

The ratio of water-insoluble particles to water soluble polymer in the composition may range from 0.05 to 100, preferably from 0.1 to 10, and most preferably from 0.25 to 3. The preferred range will depend at least in part on the molecular weight of the water soluble polymer. If a high molecular weight polymer is used (e.g. >>1.5 MM), the quantity used will typically be low (<0.1%) and the corresponding ratio of water-insoluble particles to water soluble polymer will be correspondingly high.

The amount of polymers included in the shaving compositions of the present invention will vary depending on the form of the final composition, but the amount should be a lubriciously effective amount, i.e., an amount sufficient to provide an effective lubricious layer on the skin. Typically, aqueous compositions will comprise about 0.005 to 10%, preferably about 0.01 to 5%, more preferably about 0.1 to 2%, by weight of the water soluble polymer(s). For example, aqueous shaving compositions may include about 0.05 to 3%, preferably about 0.1 to 2%, more preferably about 0.1 to 1%, by weight of the higher molecular weight polyethylene oxide and about 0.1 to 5%, and preferably about 0.1 to 2%, more preferably about 0.2 to 1.5%, by weight of the relatively lower molecular weight polyethylene oxide.

As discussed above, the shaving compositions also include a cleansing or conditioning agent for hair or skin. Suitable cleansing or conditioning agents include, for example, beard wetting agents, skin conditioning agents (e.g. vitamins A, C and E, aloe, allantoin, panthenol, alpha-hydroxy acids, phospholipids, triglycerides, botanical oils, amino acids), cleansing agents, lathering agents, emollients (e.g., fatty esters such as isopropyl myristate, decyl oleate, 2ethylhexyl palmitate, PEG-7 glyceryl cocoate, and glyceryl linoleate, propoxylated fatty ethers such as PPG-10 cetyl ether and PPG-11 stearyl ether, di- and triglycerides such as lecithin and caprylic/capric triglyceride, vegetable oils, and similar materials), humectants (e.g., glycerin, sorbitol, propylene glycol), and water dispersible or soluble surface active agents (e.g., soaps (including interrupted soaps), detergents, non-ionic, anionic and amphoteric surfactants). Other suitable cleansing and conditioning agents include skin freshening and soothing agents such as menthol, aloe, allantoin, lanolin, collagen and hyaluronic acid, fluorosurfactants, silicones (e.g. dimethicone, dimethiconol, dimethicone copolyol, stearyl dimethicone, cetyl dimethicone copolyol, phenyl dimethicone, cyclomethicone, etc.), and other vitamins (including vitamin precursors and derivatives) such as tocopherol acetate and vitamin A palmitate.

Advantageously, other cosmetic ingredients may be added to improve the application aesthetics and/or achieve other shave benefits. For example, the composition may include one or more of the following components: foaming agents, gelling or thickening agents, propellants or self-foaming agents, fragrances, colorants, antioxidants, preservatives, and other ingredients used in shaving compositions.

The shaving compositions of the present invention may be formulated as simple aqueous solutions that can be applied to the face prior to shaving. However, preferably the composition will be in the form of a shaving lotion, cream, foam or gel. Such a formulation will typically comprise, by weight, about 60% to 95%, preferably about 70% to about 90%, water and about 3% to 25%, preferably about 5% to 20%, of a water dispersible (or soluble) surface active agent.

The surface active agent may include one or more water soluble soaps (including interrupted soaps), detergents, anionic surfactants, amphoteric surfactants and/or non-ionic surfactants. Naturally, of course, the shaving composition may contain a variety of well-known cosmetic ingredients which are typically used to enhance the performance attributes and aesthetics thereof.

A preferred shaving composition of the present invention comprises about 60% to 95% water, about 3% to 25% of a water dispersible surface active agent, about 0.01% to 2.0% of water insoluble polymer particles, and about 0.005% to 10%, preferably about 0.01% to 5%, of at least one water soluble polymer, preferably two polymers.

The water dispersible surface active agent may comprise a soap, a detergent, an anionic surfactant, a non-ionic surfactant, or a mixture of one or more of these. The soaps include, for example, the sodium, potassium and lower alkanolamine (preferably triethanolamine) salts of C10 to C20, preferably C12 to C18, fatty acids. Typical fatty acids include lauric, oleic, coconut oil, myristic, palmitic and stearic acid and mixtures thereof. The preferred fatty acids are palmitic and stearic. For purposes of the present invention, the water dispersible soaps are also intended to include the interrupted soaps such as the sodium, potassium and lower alkanolamine (preferably triethanolamine) salts of N-fatty acyl sarcosines wherein the fatty acyl moiety has 10 to 20, preferably 12 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps (including the interrupted soaps) may be utilized in pre-neutralized form (i.e., as the sodium, potassium or alkanolamine salt) or in the free acid form followed by subsequent neutralization with sodium hydroxide, potassium hydroxide and/or alkanolamine (preferably triethanolamine). In any event, the composition must contain sufficient base to neutralize or partially neutralize the soap component and adjust the pH to the desired level.

The water dispersible surface active agent may also optionally include a non-ionic, amphoteric and/or anionic surfactant. Suitable non-ionic surfactants include fatty acid ester of polyols (e.g. glyceryl mono/dioleate), polyoxyethylene ethers of fatty alcohols, acids and amides, particularly those having 10 to 20, preferably 12 to 18, carbon atoms in the fatty moiety and about 8 to 60, preferably 10 to 30, ethylene oxide units. These include, for example, PEG-150 Distearate, Oleth-20, Steareth-21, Ceteth-20, and Laureth-23. Other non-ionic surfactants include the polyoxyethylene ethers of alkyl substituted phenols, such as Nonoxynol-4 and Nonoxynol-20, fatty alkanolamides such as Lauramide DEA and Cocamide MEA, polyethoxylated sorbitan esters of fatty acids, such as Polysorbate-20, lauryl polyglucoside, sucrose laurate, and polyglycerol 8-oleate. Suitable amphoteric surfactants include, for example, the betaines and sultaines such as cocoamidopropyl betaine, coco dimethyl carboxymethyl betaine, coco sultaine and the like. Suitable anionic surfactants include, for example, the sodium, potassium, ammonium and substituted ammonium salts (such as the mono-, di- and triethanolamine salts) of C8-C22, preferably C12-C18, alkyl sulfates (e.g. sodium lauryl sulfate, ammonium lauryl sulfate), alkyl sulfonates (e.g. ammonium lauryl sulfonate), alkylbenzene sulfonates (e.g. ammonium xylene sulfonate), acyl isethionates (e.g. sodium cocoyl isethionate), acyl lactylates (e.g. sodium cocoyl lactylate) and alkyl ether sulfates (e.g. ammonium laureth sulfate). The surface active agent may typically include up to about 8% of non-ionic, amphoteric and/or anionic surfactants.

In addition to the surface active agent, the shaving composition may optionally include a variety of other well-known cosmetic ingredients generally known for use in shaving creams, foams and gels to improve the aesthetics and performance characteristics of the composition.

The shaving composition may contain about 1% to 10%, preferably about 1.5% to 7%, of a non-volatile paraffinic hydrocarbon fluid. The terms “non-volatile” and “fluid” mean that these materials are liquid at room temperature and have a boiling point above 200° C. Such hydrocarbon fluids include mineral oils and branched-chain aliphatic liquids. These fluids typically have from about 16 to about 48, preferably about 20 to about 40, carbon atoms and a viscosity of about 5 to about 100 cs., preferably about 10 to about 50 cs., at 40° C. The preferred non-volatile paraffinic hydrocarbon fluid is selected from mineral oil with a viscosity of about 10 to about 50 cs. at 40° C., hydrogenated polyisobutene with a molecular weight of about 320 to about 420, and mixtures thereof.

It may also be desirable to include a water soluble gelling aid or thickening agent in the shaving composition to improve its consistency and stability, as well as to adjust its viscosity. These may include, for example, hydroxyalkyl cellulose polymers such as hydroxyethyl cellulose and hydroxypropyl cellulose (sold under the trademarks “Natrosol” and “Klucel” respectively), copolymers of acrylic acid and polyallyl sucrose (sold under the trademark “Carbopol”), carboxymethyl cellulose, and cellulose methyl ether (sold under the trademark “Methocel”). The gelling aid or thickening agent is typically included in an amount of about 0.01% to 5%, preferably about 0.1% to 2%, by weight of the composition. The shaving composition may also include up to 8%, preferably about 2% to 6%, by weight of a fatty alcohol such as myristyl, lauryl and stearyl alcohol and octyl dodecanol. The term “fatty” is intended to include 10 to 20, preferably 12 to 18, carbon atoms.

If the shaving composition is in the form of a self-foaming shave gel, it will include a self-foaming agent which may be any volatile hydrocarbon or halohydrocarbon with a sufficiently low boiling point that it will volatilize and foam the gel upon application to the skin, but not so low that it causes the gel to foam prematurely. The typical boiling point of such an agent generally falls within the range of −20° to 40° C. Preferred self-foaming agents are selected from saturated aliphatic hydrocarbons having 4 to 6 carbon atoms, such as n-pentane, isopentane, neopentane, n-butane, isobutane, and mixtures thereof. Most preferred is a mixture of isopentane and isobutane in a weight ratio (IP:IB) of about 1:1 to about 9:1, preferably about 2:1 to about 7:1. The self-foaming agent will normally be present in an amount comprising about 1% to about 6% of the composition, preferably about 2% to about 5%.

If the shaving composition is in the form an aerosol foam, it will include a propellant of sufficient volatility or pressure to propel the shaving composition from its container and cause it to foam. Typical propellants include compressed air and, more typically, a volatile hydrocarbon or halohydrocarbon or mixture of hydrocarbons (typically with 3 to 6 carbon atoms) having a vapor pressure of 30 to 60 psig at about 20° C. A preferred propellant has the industry designation A-46 and is a mixture of n-butane, isobutane and propane with a vapor pressure of 46 psig at about 20° C. Other suitable propellants include propellant 152A and A-70. When the propellant is a volatile hydrocarbon, it typically comprises about 1% to 5%, preferably about 2% to 4%, of the composition.

One type of shaving composition of the present invention is an aerosol shaving foam which comprises, in percent by weight, about 65% to 90% (preferably about 70% to 90%) water, about 3% to 25% (preferably about 5% to 20%) of a water dispersible surface active agent, a lubriciously effective amount of a lubricating agent as described above, and propellant (preferably about 1% to 5% volatile hydrocarbon). In one embodiment, the lubricating agent will include, for example, about 0.01% to 0.8% (preferably about 0.05% to 0.4%) of a relatively high molecular weight polyethylene oxide (average molecular weight=4-5 million), about 0.01% to 0.8% (preferably about 0.05% to 0.4%) of a relatively lower weight polyethylene oxide (average molecular weight=300,000-1,000,000), and about 0.01% to 2% (preferably about 0.1% to 1%) water insoluble polymer particles (e.g., micronized PTFE particles).

Another type of shaving composition of the present invention is a non-aerosol shave gel which comprises, in percent by weight, about 65% to 95% (preferably about 70% to 90%) water, about 3% to 25% (preferably about 5% to 20%) of a water dispersible (or soluble) surface active agent, and a lubriciously effective amount of a lubricating agent. In one embodiment, the lubricating agent will include, for example, about 0.01% to 2% (preferably about 0.05% to 1%) of a relatively high molecular weight polyethylene oxide (average molecular weight=4-5 million), about 0.01% to 2% (preferably about 0.05% to 1.5%) of a relatively lower weight polyethylene oxide (average molecular weight=300,000-1,000,000), and about 0.01% to 3% (preferably about 0.1% to 1.5%) water insoluble polymer particles (e.g., micronized PTFE particles).

A further type of shaving composition of the present invention is a self-foaming shave lotion or gel which comprises, in percent by weight, about 65% to 95%, (preferably about 70% to 90%) water, about 3% to 25% (preferably about 5% to 20%) of a water dispersible (or soluble) surface active agent, a lubriciously effective amount of a lubricating agent, and about 1% to 6% (preferably about 2% to 5%) self-foaming agent (e.g., volatile hydrocarbon). In one embodiment, the lubricating agent will include, for example, about 0.01% to 2% (preferably about 0.05% to 1%) of a relatively high molecular weight polyethylene oxide (average molecular weight=4-5 million), about 0.01% to 2% (preferably about 0.05% to 1.5%) of a relatively lower weight polyethylene oxide (average molecular weight=300,000-1,000,000), and about 0.01% to 3% (preferably about 0.1% to 1.5%) water insoluble polymer particles (e.g., micronized PTFE particles).

The shaving compositions of the present invention may be packaged in any suitable dispenser normally used for dispensing shaving lotions, creams, foams or gels. These include aerosol dispensers in which the propellant is added to the same chamber as the shaving concentrate (e.g., as is typically done with shaving foams), aerosol dispensers with a barrier, such as a collapsible bag or piston, to separate the shaving concentrate from the propellant required for expulsion (e.g., as is typically done with self-foaming gels), collapsible tubes, and pump or squeeze containers. It is preferred to protect the composition from oxidation and heavy metal contamination. This can be achieved, for example, by purging the composition and container with nitrogen to remove oxygen and by utilizing inert containers (e.g., plastic bottles, aluminum cans or polymer coated or lined cans).

The present invention is also directed to an improved shaving method in which a shaving composition of the present invention is applied to an area of skin, then said area is shaved, preferably with a wet razor. In particular, the invention embraces a method of shaving comprising applying to an area of skin a shaving preparation including a lubricating agent as discussed above, and shaving said area of skin. The shaving composition may be an aqueous polymer solution, such as a shave lotion, cream, foam or gel.

The invention may be further described by the following examples in which all parts and percentages are by weight.

### EXAMPLE

A number of shaving compositions were made according to the formulations shown in the following table.

| | | | | | | | AEROSOL | | NON | |
|---|---|---|---|---|---|---|---|---|---|---|
| | AEROSOL GEL | | | | | | FOAM | | AEROSOL | |
| EXAMPLE | 1 | 2 | 3 | 4 | 5 | 6 | 7 | 8 | 9 | 10 |
| De-ionized | 75.7 | 75.65 | 75.61 | 74.91 | 76.73 | 77.40 | 83.26 | 82.49 | 77.34 | 77.74 |
| water (q.s. | | | | | | | | | | |
| .100 | | | | | | | | | | |
| Hydroxyethyl | 0.500 | 0.500 | 0.500 | 0.500 | 0.486 | 0.486 | 0.400 | 0.500 | 0.70 | 0.70 |
| cellulose | | | | | | | | | | |
| PEG-90M | 0.090 | 0.090 | 0.090 | 0.090 | 0.058 | 0.058 | 0.10 | 0.10 | 0.10 | 0.10 |
| Palmitic Acid | 7.750 | 7.750 | 7.750 | 7.750 | 7.529 | 7.529 | 6.50 | 6.85 | 8.25 | 8.25 |
| Stearic Acid | 2.600 | 2.600 | 2.600 | 2.600 | 2.526 | 2.526 | | | 2.75 | 2.75 |
| Sorbitol | 1.000 | 1.000 | 1.000 | 1.000 | 0.972 | 0.972 | 1.000 | 1.000 | 1.0 | 1.0 |
| (70%) | | | | | | | | | | |
| Glyceryl | 2.000 | 2.000 | 2.000 | 2.000 | 1.943 | 1.943 | 1.000 | 0.700 | 2.0 | 2.0 |
| Oleate | | | | | | | | | | |
| TEA 99% | 6.350 | 6.350 | 6.350 | 6.350 | 5.88 | 5.88 | 3.88 | 4.10 | 6.35 | 6.35 |
| Fragrance | 0.950 | 0.900 | 0.950 | 0.950 | 0.769 | 0.30 | 0.30 | 0.80 | 0.80 | |
| Polytetrafluoro- | 0.005 | 0.05 | 0.1 | 1 | 0.1457 | 0.146 | 0.25 | 0.25 | 0.22 | 0.22 |
| ethylene* | | | | | | | | | | |
| PEG-14M | | 0.050 | | | | | | | | |
| PEG-23M | | | | | 0.0486 | 0.0486 | | | 0.06 | 0.06 |
| Dye | 0.200 | 0.200 | 0.200 | | 0.000972 | 0.000972 | | | | |
| Vitamin E | 0.005 | 0.005 | 0.005 | 0.005 | 0.115 | 0.1635 | 0.015 | .005 | 0.435 | .035 |
| Isopentane | 2.14 | 2.14 | 2.14 | 2.14 | 2.14 | 2.14 | | | | |
| Propane | | | | | | | 0.64 | 0.64 | | |
| Isobutane | 0.71 | 0.71 | 0.71 | 0.71 | 0.71 | 0.71 | 2.60 | 2.6 | | |
| TOTAL | 100.00 | 100.00 | 100.00 | 100.00 | 100.00 | 100.00 | 100.00 | 100.00 | 100.00 | 100.00 |

*Micro Powders, Inc.

These examples represent various aerosol and non-aerosol shaving gels with different additives and/or different use levels.

A number of embodiments of the invention have been described. Nevertheless, it will be understood that various modifications may be made without departing from the spirit and scope of the invention.

For example, while preferred shaving preparations include a lubricating agent that contains both water insoluble polymer particles and a blend of two or more water soluble polymers, in other embodiments the blend of water soluble polymers may be omitted and the water insoluble polymer particles used alone. In other embodiments, the water insoluble polymer particles may be omitted, and a blend of water soluble polymers having different molecular weight distributions used alone. Other combinations may also be employed, for example water insoluble polymer particles may be used with a single water soluble polymer. Accordingly, other embodiments are within the scope of the following claims.

1. A method of shaving comprising applying to an area of skin a shaving composition and shaving said area of skin, wherein the shaving composition comprises an aqueous solution including a cleansing or conditioning agent for hair or skin, water-insoluble lubricating polymer particles comprising polytetrafluoroethylene, and a water soluble polymer, the shaving composition being substantially free of anionic polymers.

2. The method of claim 1 wherein the water soluble polymer comprises polyethylene oxide.

3. The method of claim 1 wherein the shaving composition is in the form of a shaving lotion, cream, foam or gel.

4. The method of claim 1 wherein the lubricating polymer particles and water soluble polymer are present in a ratio of about 0.25:1 to about 3:1.

5. The method of claim 1 wherein the shaving composition includes from about 0.005% to 2% of said lubricating polymer particles.

6. The method of claim 1 or 5 wherein the shaving composition includes from about 0.01 to 2.0% of said water soluble polymer.

7. The method of claim 1 wherein the polymer particles have an average particle size of from about 1 μm to 100 μm.

8. The method of claim 1 wherein the shaving composition further comprises from about 3 to 25% of a water dispersible or water soluble surface active agent.

9. The method of claim 1 wherein the cleansing or conditioning agent is selected from the group consisting of beard wetting agents, skin conditioning agents, cleansing agents, lathering agents, emollients, humectants, skin freshening and soothing agents, silicones, vitamins, and water dispersible or soluble surface active agents.

10. The method of claim 1 wherein the water soluble polymer is selected from the group consisting of polyethers, vinyl polymers, vinyl copolymers and terpolymers, natural polymers, and mixtures thereof.

11. The method of claim 1 wherein the water soluble polymer comprises a blend of polyethylene oxide and a water soluble natural or synthetic gum.

12. The method of claim 10 or 11 wherein the shaving composition includes from about 0.01 to 2.0% of said water soluble polymer.

13. The method of claim 10 wherein said water soluble polymer has a molecular weight of at least one million.

14. A shaving composition comprising
water,
a cleansing or conditioning agent for hair or skin, and
a lubricating agent comprising
(a) lubricating polymer particles comprising PTFE, and
(b) a water soluble polymer
wherein the lubricating agent is substantially free of anionic polymers.

15. The shaving composition of claim 14 wherein the water soluble polymer is selected from the group consisting of polyethers, vinyl polymers, vinyl copolymers and terpolymers, natural polymers, and mixtures thereof.

16. The shaving composition of claim 14 wherein the water soluble polymer comprises polyethylene oxide.

17. The shaving composition of claim 14, 15 or 16 wherein the shaving composition includes from about 0.01 to 2.0% of said water soluble polymer.

18. The shaving composition of claim 15 wherein said water soluble polymer has a molecular weight of at least one million.

19. The shaving composition of claim 14 wherein the water soluble polymer comprises polyethylene oxide.

20. The shaving composition of claim 14 wherein the shaving composition is in the form of a shaving lotion, cream, foam or gel.

21. The shaving composition of claim 14 wherein the lubricating polymer particles and water soluble polymer are present in a ratio of about 0.25 to 3.

22. The shaving composition of claim 21 wherein the shaving composition includes from about 0.01 to 2% of said water soluble polymer.

23. The shaving composition of claim 14 wherein the polymer particles have an average particle size of from about 1 μm to 100 μm.

24. The shaving composition of claim 14 or 22 wherein the shaving composition includes from about 0.005% to 2% of said lubricating water-insoluble polymer particles.

25. The shaving composition of claim 14 wherein the shaving composition further comprises from about 3 to 25% of a water dispersable or water soluble surface active agent.

26. The shaving composition of claim 14 wherein the cleansing or conditioning agent is selected from the group consisting of beard wetting agents, skin conditioning agents, cleansing agents, lathering agents, emollients, humectants, skin freshening and soothing agents, silicones, vitamins, and water dispersible or soluble surface active agents.

27. The shaving composition of claim 14 in the form of a lotion, cream, foam or gel.

28. The shaving composition of claim 14 additionally comprising a volatile self-foaming agent.

29. The shaving composition of claim 14 wherein the water soluble polymer comprises a blend of polyethylene oxide and a water soluble natural or synthetic gum.

30. A shaving composition comprising:
water,
a cleansing or conditioning agent for hair or skin, and
a lubricating agent comprising
(a) from about 0.005% to 2% of lubricating polymer particles comprising PTFE, and
(b) from about 0.01 to 2.0% of a water soluble polymer selected from the group consisting of polyethers, vinyl polymers, vinyl copolymers and terpolymers, natural polymers, and mixtures thereof.

31. A shaving composition comprising:
water,
a cleansing or conditioning agent for hair or skins selected from the group consisting of beard wetting agents, skin conditioning agents, cleansing agents, lathering agents, emollients, humectants, skin freshening and soothing agents, silicones, vitamins, and water dispersible or soluble surface active agents, and
a lubricating agent comprising
(a) from about 0.005% to 2% of lubricating polymer particles comprising PTFE, and
(b) from about 0.01 to 2.0% a water soluble polymer;
wherein components (a) and (b) are provided in a ratio of about 0.25 to 3.
